(19) United States
(12) Patent Application Publication
(10) Pub. No.: US 2010/0173842 A1
(43) Pub. Date: Jul. 8, 2010

(54) VACCINE FOR STAPHYLOCOCCAL INFECTIONS

(75) Inventors: Krishna Murthy ELLA, Hyderabad (IN); Kandaswamy SUMATHY, Hyderabad (IN); Krishna Mohan VADREVU, Hyderabad (IN)

Correspondence Address: MATTHIAS SCHOLL, 14781 MEMORIAL DRIVE, SUITE 1319, HOUSTON, TX, 77079, US

(73) Assignee: BHARAT BIOTECH INTERNATIONAL LIMITED, Hyderabad (IN)

(21) Appl. No.: 12/575,667

(22) Filed: Oct. 8, 2009

Related U.S. Application Data
(63) Continuation-in-part of application No. 12/067,458, filed on Aug. 18, 2008, now abandoned.

(30) Foreign Application Priority Data
Jul. 14, 2005 (IN) 940/CHE/2005

(51) Int. Cl. A61K 38/16; C07K 14/31; C12N 15/63; C12P 21/00; A61K 31/7052; G01N 33/53; A61P 31/04

(52) U.S. Cl. 514/12; 530/350; 435/320.1; 435/69.1; 514/44 R; 436/501

(57) ABSTRACT

The invention relates to a method of preparation and use of a polypeptide vaccine formulation for prevention and control of Staphylococci mediated infections in human, bovine and other mammals, using recombinant DNA technology.

### CROSS-REFERENCE TO RELATED APPLICATIONS

This application is a continuation-in-part of U.S. application Ser. No. 12/067,458, which is a National Stage Appl. of International PCT Appl. No. PCT/IN2006/000246 with an international filing date of Jul. 13, 2006, which is based on Indian Appl. No. 940/CHE/2005, filed Jul. 14, 2005. The contents of all of the aforementioned applications are incorporated herein by reference in their entirety.

### BACKGROUND OF THE INVENTION

1. Field of the Invention

The invention relates to a method of preparation and use of polypeptide vaccine formulation for prevention and control of Staphylococci mediated infections in human, bovine and other mammals, using recombinant DNA technology.

2. Description of the Related Art

The gram-positive bacteria Staphylococci normally inhabiting skin and mucous membranes in humans, gains access to internal tissues during injury/surgery and cause infection. The bacterium has a characteristic propensity of invading skin and adjacent tissues at sites of prosthetic medical devices, including intravascular catheters, cerebrospinal fluid shunts, hemodialysis shunts, vascular grafts, and extended-wear contact lenses (Lowy 1998, Foster 2004).

The important pathogens in the Staphylococcus genus are the coagulase positive Staphylococcus aureus and the coagulase negative Staphylococcus epidermidis. They cause a variety of diseases ranging from skin diseases, wound sepsis, and mastitis to life threatening endocarditis, osteomyelitis, and chronic lung infections in human and animals. They are responsible for 1-9% cases of bacterial meningitis and 10-15% cases of brain abscesses (Foster 2004). Mortality due to Staphylococci bacteremia is approximately 20-50% due to emergence of drug resistance, including resistance to Methicillin and Vancomycin (Enright 2002, Mongodin 2003). Staphylococci are the leading cause of community acquired and nosocomial (hospital-acquired) infections associated with prolonged hospitalization (Franklin 2003).

Staphylococcus virulence is multifactorial, mediated by a number of virulence factors such as alpha, beta, gamma and delta-toxins, toxic shock syndrome toxin (TSST), enterotoxins, leucocidin, proteases, staphylokinase, coagulase, and clumping factor (Jin 2004, Martin 2003). To initiate invasive infection, Staphylococcus adheres to extracellular matrix substrates and eukaryotic cells by virtue of different surface proteins (“adhesins”) (Peacock 2002). These surface proteins produced by Staphylococcus are designated as MSCRAMMs (microbial surface components recognizing adhesive matrix molecules). These bind specifically to biological substrates such as serum proteins, IgG, fibronectin (Fn), fibrinogen (Fg), vitronectin, thrombospondin, thereby masking the bacterium from host's immune system (Hartford 1999, Harris 2002). These surface proteins can also bind to collagen, laminin, glycosaminoglycans, and elastin from extra cellular matrix (Snodgrass, 1999) of a wounded tissue or an injured vessel wall, bone sialoprotein, bind to platelets (Siboo 2001) and to non-biological substrates like medical devices (catheters, shunts, pacemakers). All these interactions contribute to colonization of host tissues, but the importance of each of these binding functions in different infections is still unclear (Theresa 1999).

Continued emergence of resistance to antibiotics has incited the need for alternative strategies for the prevention and treatment of Staphylococcal diseases. Vaccination has proved relatively unsuccessful against the common mammalian commensal bacteria Staphylococci, despite almost a century of experimentation (Michie 2002). Several researchers have attempted to produce killed, attenuated, fixed, or lysed S. aureus vaccines and/or to isolate capsular polysaccharides or cell wall components, which will induce immunity to S. aureus. Nevertheless, none of these attempts have been successful. Toxoids induced high antibody titers in several studies, but proved to be unsuccessful vaccine candidates as they induced adverse reactions. The development of polysaccharide antigenic components of the Staphylococcal capsule is complicated by the myriad of strains prevalent in the community.

Ali Fattom et al (1996) (Nabi Pharmaceuticals) developed a vaccine (StaphVAX) by linking the polysaccharides type 5 and type 8 purified from S. aureus to a carrier protein (a nontoxic form of Pseudomonas aeruginosa exotoxin) and demonstrated 56% protection against S. aureus in patients receiving hemodialysis (Shinefield H. 2002). Like previous vaccines, this vaccine also could not provide prolonged protection against invasive Staphylococci even though it had greater efficacy and fewer side effects. 1 ng-Marie Nilsson et al (1998) demonstrated that vaccination with a recombinant version of the collagen adhesin, protected mice against heterologous challenge of S. aureus. Dr Gerald Pier et al (1999) demonstrated vaccination with purified surface polysaccharide PNSG (poly-N-succinyl beta-1-6 glucosamine) on S. aureus protected mice against a challenge of S. aureus.

But as of present, there is no vaccine in the market providing full protection against Staphylococcal infections (Michie 2002). Vaccine strategies targeting microbial surface components recognizing adhesive matrix molecules (MSCRAMM) are viable approaches to impede bacterial adherence, prevent colonization, and minimize hematogenous dissemination, thereby halting the inception and progression of infection.

### SUMMARY OF THE INVENTION

Therefore, in search of a novel vaccine candidate, the surface proteins of S. aureus and S. epidermidis were analyzed in-silico. As adherence is the critical step in pathogenesis, the available completed genome sequences in public database of S. aureus and S. epidermidis strains were analyzed in-silico for previously unknown/uncharacterized Staphylococcal adhesins. Here we report immunization of mice with recombinant protein from S. aureus having role in adhesion and autolytic property, giving protection against heterologous challenge of S. aureus and S. epidermidis.

The following groups of patients could benefit from vaccine against S. aureus and S. epidermidis: surgical patients undergoing lengthy cardiac and orthopedic procedures, trauma and burn patients, patients receiving an implanted medical device or prosthetic, newborns whose immune systems are not yet developed, individuals in long-term care, and kidney dialysis patients, and others.

Accordingly, the invention provides an antigenic composition of the protein comprising of amino acid sequence of SEQ ID NO: 2 or comprising any one of the mutants and variants thereof, whereas the mutants and variants include at least one of the following: deletion(s), and/or domain replacement(s) of the amino acids responsible for protein-protein interactions, and/or cell wall targeting and to be used as a vaccine for prevention and control of Staphylococcal infections:

A composition as claimed in claim 1 wherein the amino acid sequence consists of SEQ ID NO: 3,

A composition as claimed in claim 1 wherein the amino acid sequence consists of SEQ ID NO: 4,

A composition as claimed in claim 1 wherein the amino acid sequence consists of SEQ ID NO: 5,

A composition as claimed in claim 1 wherein the amino acid sequence consists of 3 LysM domains and one CHAP domain,

A recombinant DNA construct comprising: (i) a vector, and (ii) at least one nucleic acid fragment encoding amino acid sequences as above or their mutants and/or variants thereof.

In certain embodiments, the invention relates to a vaccine for Staphylococcal infection. The invention provides vaccine for Staphylococcal infection in mammals in general and human beings and or cattle in particular. The invention also provides a recombinant and highly immunogenic protein, more specifically surface antigen from S. aureus, which can be used as an antigenic molecule in a vaccine compostion. Further, the said protein is having staphylolytic activity. The recombinant protein of the invention discussed here comprises repeats of LysM domains, which exhibit peptidoglycan binding property and CHAP (cysteine, histidine-dependent amido hydrolases/peptidases) domain that exhibits peptidoglycan cleaving property. Further, the invention provides a method for isolation and purification of the said recombinant protein. The composition of the protein in pharmacologically and pharmaceutically acceptable carrier/adjuvant/stabilizer is also given. The pharmaceutical composition of the said protein is immunogenic and is effective as a vaccine against Staphylococcal infections in the animal model. An immunodiagnostic method was also developed for the diagnosis of Staphylococcal infections. The protein is a potential candidate for prophylactic and diagnostic purposes.

### BRIEF DESCRIPTION OF THE DRAWINGS

The invention is described hereinbelow with reference to accompanying drawings, in which:

FIG. 1 shows the shading of conserved amino acids of Aaa protein of S. aureus (SEQ ID NO.2) and the homologues gene Aae of S. epidermidis genes (SEQ ID NO. 6) done by TEXSHADE (Biology WorkBench) at 50% identity threshold;

FIG. 2 shows domain analysis of Aaa gene by Pfam (http://www.sanger.ac.uk/Software/Pfam/). This protein has 3 LysM domains between residues 4-47, 68-111 and 135-178 and 1 CHAP domain between residues 191-311;

FIG. 3 represents analysis of purified protein in SDS PAGE (15%); lane 1 shows the migration pattern of protein molecular weight markers and lane 2 shows purified protein;

FIG. 4 shows agarose gel showing the presence of autolysin adhesin gene in S. aureus and S. epidermidis (A); S. aureus clinical isolates and ATCC strains contain the Aaa gene needed for autolysin adhesin synthesis; PCR performed on chromosomal DNA from 4 clinical isolates and 3 ATCC stains of S. aureus; lanes 1-4 clinical isolates from hospitalized patients isolated from bacteremia, intra vascular catheter, kidney dialysis patient, and femur, respectively, and were resistant to Methicillin; lane 5, ATCC 25923; lane 6, ATCC 33591; lane 7, ATCC 29737 (B); S. epidermidis clinical isolates and ATCC strains contain the Aae gene needed for autolysin adhesin synthesis; PCR performed on chromosomal DNA from three clinical isolates and two ATCC stains of S. epidermidis; lane 1-3 clinical isolates from hospitalized patients isolated from bacteremia, post operative wound infection, endocarditis respectively; lane 5, ATCC 12228; lane 6, ATCC 35547; lane 7, molecular weight markers; primers were designed to amplify a 930 base pair fragment corresponding to mature protein;

FIG. 5 is a zymogram showing the bacteriolytic activity of Aaa. SDS-PAGE of His 6-tagged Aaa purified from E. coli (lane 2 and 3); the separation gel (10%) contained heat-inactivated S. aureus cells (0.2%) in lane 2 and heat inactivated S. epidermidis cells (0.2%) in lane 3 as a substrate for autolysin; bacteriolytic activity is visible as a clear zone in both S. aureus and S. epidermidis, after incubation in phosphate buffer at 37° C.; the arrow indicates Aaa-associated bacteriolytic activity; molecular weight marker is shown in lane 1;

FIG. 6 shows Western blots of purified autolysin adhesin of S. aureus with pooled sera of mice infected with S. aureus and pooled sera of human infected with S. aureus; lane 1, the positions of molecular mass markers (kD); lane 2, pooled sera patient; lane 3, pooled sera healthy adult; lane 4, negative control—pooled sera of children (6 to 18 months) showing no band; lane 5, pooled sera of mice infected with S. aureus; lane 6, pooled healthy mice sera showing no band; bands indicate production of antibodies against autolysin adhesin of S. aureus when human or mice exposed to S. aureus; arrow indicates a 34 kD band corresponding to autolysin adhesion;

FIG. 7 represents protective efficacy of recombinant protein; active immunization of mice with recombinant protein provides protection against challenge with S. aureus and S. epidermidis; bars indicate log mean CFU per pair of kidneys in mice immunized with protein Aaa (solid bars) or with an irrelevant protein BSA (speckled bars); challenge species and doses in CFU per mouse is given below each group; N=10 mice per group; the challenging strains were S. aureus ATCC 25293 in group A; S. aureus ATCC 33591 in group B; Clinical isolate of MRSA (Femur bone) in group C; S. epidermidis ATCC 12228 in group D; and

FIG. 8 shows IgG antibody titers obtained in pooled sera of mice (8) immunized intraperitoneally with protein Aaa; animals were immunized with 2 doses of 100 μg of protein; blood samples were obtained at 2 weeks interval for 1-9 weeks after the final immunization.

### DETAILED DESCRIPTION OF THE INVENTION

The invention relates to development of a recombinant protein vaccine from S. aureus, useful for inducing immunity for the prevention and treatment of Staphylococcal infections. The invention further relates to isolation of the protein and purification of the said protein for immunization against the infections associated with S. aureus and S. epidermidis. The invention reveals that protein is also useful for producing antibodies for therapeutic and diagnostic purposes. The instant invention is based on the finding that the protein identified and expressed in S. aureus strain having role in adhesion and is highly immunogenic. The invention further provides method of using purified protein as suitable vaccine candidate.

The above gene and protein sequences have been engineered to reduce or abolish potential protein-protein interactions with cellular and extracellular proteins such as the sequences described in SEQ ID NO: 4 and in SEQ ID NO: 5. The invention also provides pharmaceutical compositions of protein that can be used as vaccine. The invention also describes a method of eliciting antigen specific immune response by immunization

The invention is based on cloning and expression of gene Aaa (SEQ ID NO: 1) encoding a adhesin/autolysin. The gene encodes a protein of 334 amino acids, with 3 repeats of LysM domains that exhibit peptidoglycan binding property, one CHAP domain exhibiting peptidoglycan cleaving property and a typical Gram-positive signal peptide suggesting that the protein is a cell wall protein. Because bacterial adherence is the first critical step in the development of most infections, it is an attractive target for the development of novel vaccines. To determine if adhesion based vaccine could prevent S. aureus infection, mice were actively immunized with recombinant protein and challenged intravenously and intraperitoneally with S. aureus. And the immunized mice showed reduced or no colony forming units when kidneys were processed.

An ELISA method has been developed for the assay of the protein and could be used as diagnostic method for the detection of the antigen or the antibodies to this protein in infected samples.

The following figures and examples are included for purposes of illustration and are not intended to limit the scope of the invention.

### Example 1

### In-Silico Analysis

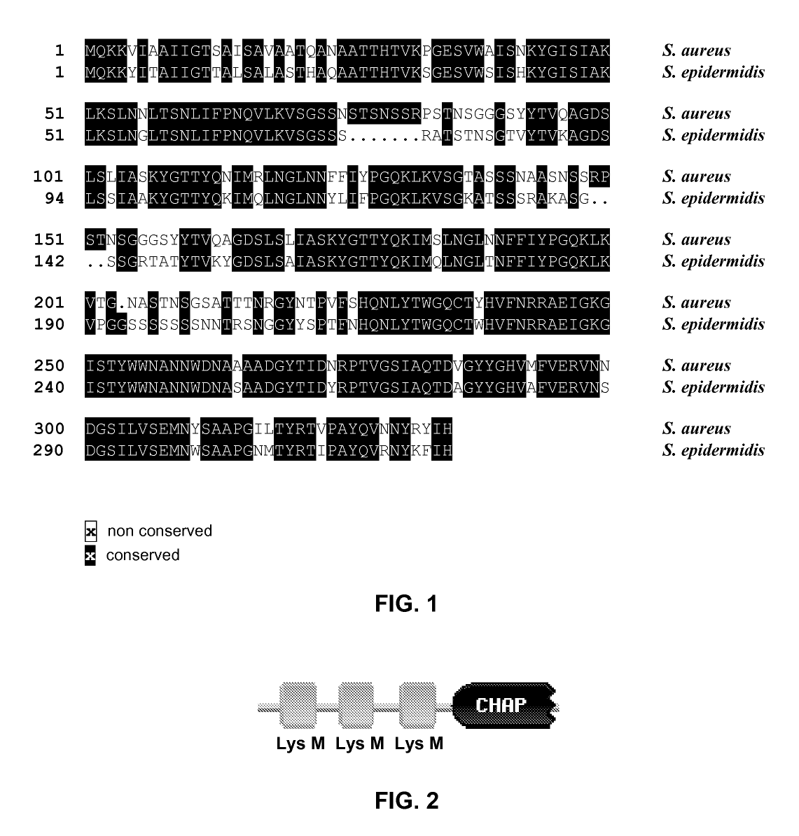

The nucleotide and amino acid sequences of both Aaa (Autolysin/adhesin of S. aureus—SEQ ID NO: 2) and Aae (Autolysin/adhesin of S. epidermidis SEQ ID NO. 6) were aligned and compared by CLUSTALW and TEXSHADE at Biology Workbench 3.2 (http://workbench.sdsc.edu/) and showed they are very similar as shown in FIG. 1. The Aaa amino acid sequence was submitted to Pfam version 17.0 (http://www.sanger.ac.ulc/Software/Pfam/) for domain analysis. Pfam domain analysis showed Aaa protein contains 3 repeats of LysM domains and 1 CHAP domain as shown in FIG. 2. The LysM (lysin motif) domain is about 40 residues long and present between the residues 4-47, 68-111 and 135-178. It is found in a variety of enzymes involved in bacterial cell wall degradation and has a general peptidoglycan binding function. The CHAP domain (cysteine, histidine-dependent amido hydrolases/peptidases) is about 120 residues long and present between residues 191-310. CHAP domain is involved in amidase function and many of the proteins having CHAP domain are involved in cell wall metabolism of bacteria. The servers Signal P 3.0 (http://www.cbs.dtu.dk/services/SignalP/), Target P 1.1 server (http://www.cbs.dtu.dlc/services/TargetP/) and PSORT version 6.4 (http://www.psort.org/) predicted protein Aaa has signal peptide of 25 residues and is located on cell wall.

### Example 2

### Bacterial Strains, Growth Conditions, and Vectors

E. coli strain DH5α was used for DNA manipulations and E. coli vector pET11b used for cloning and expression of the autolysin adhesin gene. The recombinant proteins were expressed in E. coli BL21 DE3 RIL. Staphylococci strains used in animal experiments were S. aureus (MSSA) ATCC 25923, S. aureus (MRSA) ATCC 33591, clinical isolate of S. aureus (Methicillin resistant—MRSA) from the femur of a hospitalized patient and S. epidermidis ATCC 12228. These S. aureus and S. epidermidis strains were cultured on blood agar for 24 h, then grown in tryptic soy broth containing 5% filtered serum till late log phase, harvested, washed, diluted in PBS to an appropriate concentration, and viable counts were determined by pour-plate method for inoculation in mice. The E. coli strains containing the pET15b or pET11b vectors were selected on Luria-Bertani (LB)-broth/agar containing 50 μg/mL ampicillin. S. aureus and S. epidermidis strains were grown in Vogel Johnson agar containing 0.1% potassium tellurite.

### Example 3

### Cloning and Sequencing of the Gene Encoding Protein Antigen

All DNA manipulations were performed using standard methods. Genomic DNA was isolated from S. aureus (ATCC 25293) according to Lindberg et al (1972). Oligonucleotides were designed to amplify the gene fragment corresponding to mature protein of S. aureus autolysin adhesin (Aaa) gene (Accession no AJ250906.1 gi|122217974; contained within SEQ ID NO: 1; the mature protein sequence is SEQ ID NO:3). The sequence of the forward primer used for amplification by PCR is: 5′CGAGCTCCATATGGCTACAACTCACACAGTAAAAC3′ and reverse primer sequence: 5′CGCTCGAGGGATCCTTATTAGTGATGGTGATGGTGATGGTGAATATATCTAT AATTATTTAC 3′. Nucleotide sequence corresponding to 6 Histidine tag was included in reverse primer. The amplified gene product was purified from agarose gel, digested with the restriction enzymes Nde1 and BamH1, and ligated by T4 DNA ligase into pET11b vector cleaved with the same restriction enzymes. The ligated vector was transformed into E. coli DH5α strain by CaCl₂ method. Clone was confirmed by DNA sequencing by dideoxy chain termination method using the ABI PRISM 310 DNA sequencing machine. The PC-gene program (Intelligenetics) was used for the handling of the sequences. Plasmid containing Aaa gene was isolated and transformed into E. coli BL21 (XDE3) RIL strain for expression of the target protein.

The gene encoding the protein sequences described in SEQ ID NO: 4 and SEQ ID NO: 5 were designed after deletion of the potential protein-protein interaction sites of the Aaa gene encoding the protein of SEQ ID NO: 3. The gene encoding protein sequences as in SEQ ID NO: 4 and SEQ ID NO: 5 were synthesized at GenScript Corporation, USA. The ORF encoding the engineered proteins have been cloned into EcoRI and BamH1 site of pGS 100 vector under the control of heat inducible promoter and have been transformed in E. coli BL21 (2DE3) RIL.

### Example 4

### Protein Expression and Purification

Overnight cultures of E. coli BL21 DE3 RIL cells harboring the recombinant plasmid pET111b were diluted 1:50 in 1 liter of Luria Broth containing 50 μg/mL ampicillin. E. coli cells were grown at 37° C. with shaking to get an A600 of 0.6, whereupon the expression of the target protein by T7 polymerase was induced by the addition of isopropyl-1-thio-b-D-galactopyranoside (IPTG) to a final concentration of 1 mM. Cells were harvested after 4 hr by centrifugation at 8,000 rpm for 10 min. The bacterial pellets were resuspended in buffer A (50 mM Phosphate Buffer, 0.5 M NaCl, pH 8.0, 4 M Urea, 1% Triton X 100, 1 mM PMSF). The cells were lysed by sonication at 15 microns amplitude for duration of 60 sec with an interval of 60 sec on ice for 30 cycles and the bacterial lysates were centrifuged at 12,000 g for 30 min to remove bacterial debris. As the target protein formed inclusion bodies, the cell lysate pellet were washed twice with the same buffer excluding PMSF and washed twice with the same buffer excluding Urea and Triton X 100 to remove them.

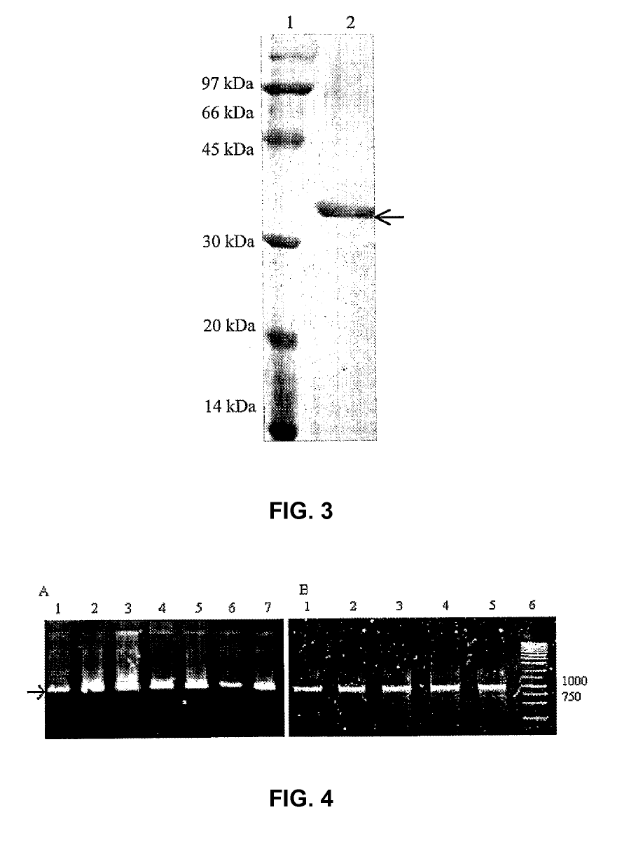

The pellet containing target protein was solubilized by suspending it in 10 volumes of 50 mM Phosphate Buffer, 0.5 M NaCl, 6 M Urea, pH 8.0, and kept on stirrer. After 4 hrs of solubilization, centrifugation was carried out at 12,000 rpm for 30 min. The supernatant containing soluble proteins was filtered through a 0.4 μm membrane and retained for further purification. The recombinant proteins were purified by immobilizing on Ni-NTA metal affinity chromatography. A column containing Ni-NTA matrix, connected to a FPLC system, was equilibrated with buffer A containing 50 mM Phosphate Buffer, 0.5 M NaCl, 6 M Urea, pH 8.0. After equilibration, the supernatant was applied to the column and the column was washed with 10 bed volumes of buffer. Subsequently, the column was eluted with buffer B containing, 50 mM Phosphate Buffer, 0.5 M NaCl, 6M Urea, pH 8, 20-200 mM imidazole. The elutions were monitored for protein by checking absorbance at 280 nm and peak fractions were analyzed by sodium dodecyl sulfate-polyacrylamide gel electrophoresis (SDS-PAGE) (FIG. 3).

### Example 5

### Occurrence of Autolysin Adhesin in S. aureus and S. epidermidis

The presence of the autolysin adhesin gene in various clinical strains of S. aureus and S. epidermidis was determined by PCR. The clinical strains were isolated from the patients suffering from sepsis, device associated infections, skin infections, renal dialysis infections, etc. The protein autolysin (Aaa) adhesin was found to be present in all the six strains of S. aureus completed genome sequences and in two strains of S. epidermidis completed genome sequences. Presence of the autolysin adhesin gene in the clinical isolates of S. aureus and S. epidermidis was confirmed by PCR as shown in FIG. 4. This shows that the autolysin adhesion (Aaa) protein is expressed in majority of the S. aureus and S. epidermidis strains.

### Example 6

### Assay of Peptidoglycan Hydrolytic Activities—Zymographic Assay

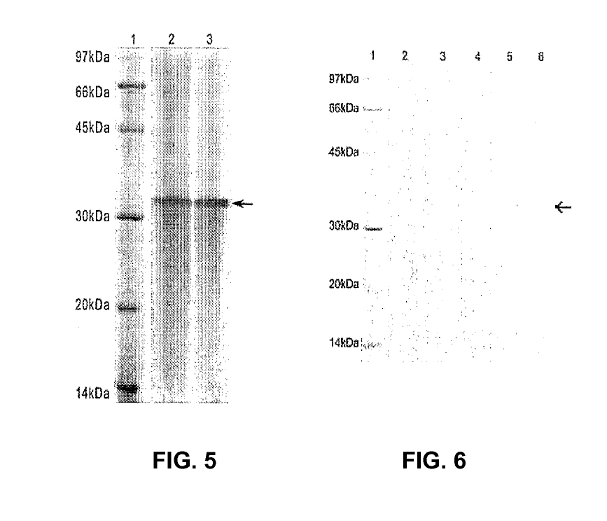

The staphylolytic activity of the Aaa protein was determined by performing a zymogram on a 10% SDS-PAGE gel as per the method described earlier with slight modifications. Briefly, 12% SDS-polyacrylamide gel was prepared containing 0.2% (w/v) heat killed S. aureus cells as substrate. The recombinant purified protein was loaded and electrophoresis was carried out at 20 mA constant current using a vertical slab gel electrophoresis assembly (Hoefer miniVE) at 4° C. Following the electrophoresis, the gel was washed thoroughly with cold distilled water containing 0.1% TritonX 100 and the gel was incubated overnight at 37° C. in 0.1 M Tris-HCl (pH 8.0) buffer. Similar assay was carried out with S. epidermidis as a substrate. Lytic bands in the translucent gel were visualized as clear bands against a blue background in an indirect light as shown in FIG. 5.

### Example 7

### Immunodiagnostic Method for the Detection of Staphylococcal Infections

ELISA

Sera from mice and humans were tested for antibodies against Aaa autolysin adhesin recombinant protein by enzyme-linked immunosorbent assay (ELISA). Microtiter wells were coated with purified protein (1 mg/mL) in 100 μL coating buffer (100 mM sodium carbonate, pH 9.2) per well and incubated overnight at 40° C. Additional protein binding sites were blocked with 2% (wt/vol) bovine serum albumin (BSA) in 200 μL/well of in phosphate-buffered saline (PBS-10 mM sodium phosphate, pH 7.4, containing 0.13 M NaCl) at room temperature for 1 hr and washed five times with PBST (0.1% Tween 20 in PBS). 100 μL of mice and human sera specimens diluted in PBST were added to different wells and incubated for 1 hr at 37° C. Unbound antibody was removed by washing the wells five times with PBST. For detection of bound antibody, 100 μL of horse radish peroxidase-conjugated goat anti-mouse IgG antibody diluted to 1:8,000 in PBST and 100 μL of horse radish peroxidase-conjugated goat anti-human IgG antibody diluted to 1:8,000 in PBST were added in respective wells and incubated for 1 hr at 37° C. After washing the wells, antigen-antibody complexes were quantified by measuring the conversion of the substrate o-phenylenediamine dihydrochloride (OPD) and H₂O₂, to colored product by the conjugated enzyme, at OD 492 nm on a micro-plate reader (Bio-Rad). Both groups of infected mice and human showed positive results in ELISA. This indicates antibodies against the protein autolysin/adhesin of S. aureus would be produced in-vivo, and is immunogenic.

### Example 8

### Western Blot

Recombinant protein was run on a 12% SDS-PAGE under reducing conditions and then electroblotted onto nitrocellulose membrane for 2 h at 200 mA using transfer buffer, CAPS buffer, pH 8.3. The membrane was then treated with a solution containing 5% (wt/vol) dried skim milk in PBS for 1 hr, followed by three washing with PBS and then incubation with high titer sera raised against the protein in mouse diluted 200 fold in PBS containing 0.05% Tween 20 (positive control), and pooled sera from S. aureus infected mice, pooled sera from S. aureus infected human and control mouse and human sera for 1 hr at 37° C. Membranes were then washed three times in PBST and subsequently incubated for 1 hr at 37° C. in 2.000-fold-diluted horseradish peroxidase-conjugated goat anti-mouse IgG in PBST, horseradish peroxidase-conjugated goat anti-human IgG in PBST, respectively. After washing, the membrane was treated with chromogenic substrate diamino benzedene (DAB) and H₂O₂. Control mouse and control human sera did not show any bands whereas positive control and S. aureus infected mice and human showed positive bands corresponding to the target protein as shown in FIG. 6. This indicates that antibodies are produced in mice and humans, against the protein when infected with S. aureus.

### Example 9

### Opsonophagocytic Assay

To determine whether antibodies produced against protein Aaa are effective in mediating the killing of S. aureus, an in vitro opsonization assay was done. Assay was done by a modified protocol of McKenney D 2000. Purified protein Aaa was injected into rabbit to get hyperimmune sera, a rich source of antibodies against the protein. Polymorphonuclear neutrophils were prepared from fresh blood collected from healthy adult rabbit. A total of 25 mL rabbit blood was mixed with an equal volume of dextran-heparin-sulfate buffer (20 g of Dextran 500/liter, 65.6 g of heparin sulfate/liter, 9 g of sodium chloride/liter) and incubated at 37° C. for 1 h. The upper layer containing leukocytes was collected, and hypotonic lysis of the remaining erythrocytes was accomplished by resuspension in 1% NH₄Cl. Subsequent wash steps were performed with RPMI with 15% fetal bovine serum. The polymorphonuclear neutrophil count was adjusted to 4×10⁶ neutrophils per mL. The complement source (guinea pig complement) was adsorbed with S. aureus to remove antibodies that could react with the target strain. After overnight growth in tryptic soy broth, S. aureus cells were centrifuged, the pellet resuspended in 1 mL of PBS. The opsonophagocytic assay was performed with 100 μL of leukocytes, 100 μL of bacteria (adjusted to 2×10⁷/mL PBS), 100 μL of the high titer serum dilution, and 100 μL of the complement source. The reaction mixture was incubated on a rotor rack at 37° C. for 90 min; samples were taken at time zero and after 90 min. Each tube was sonicated for 5 sec at 4 W and then diluted in tryptic soy broth containing 0.5% Tween and plated onto Vogel Johnson agar plates. Tubes lacking any serum and tubes with normal rabbit serum were used as controls. The assay was done by test serum showed reduced number of colonies compare to control assay. These showed antibodies against protein Aaa are effective in mediating the killing of S. aureus by phagocytes.

### Example 10

### Development of Vaccine Formulation and Efficacy Studies

The recombinant purified protein along with above mentioned PBS, adjuvant is mixed with at least one of the following stabilizers used in the concentration range of 0.05% to 5%, such as polyols (mannitol, sorbiltol, glycerol), sugars (lactose, trehalose, sucrose), human serum albumin, amino acids (glutamate, arginine, histidine).

### Immunization and Challenging of Mice

Purified, filter sterilized recombinant autolysin adhesin protein (final concentration of 0.2 mg/mL) and BSA of same concentration was suspended in sterile PBS (Phosphate Buffered Saline—10 mM Phosphate Buffer containing 0.13 M Sodium Chloride, pH 7.4) containing 0.5 mg/mL aluminum hydroxide (an adjuvant). Vaccine formulation comprising 500 μL of the emulsion containing the purified protein (1-1000 micrograms) as antigen was injected into mice (4 groups of mice containing 18 in each groups A, B, C, D) intraperitonealy (i.p.) on day 0 and 500 μL of BSA suspension injected in to 4 groups of mice containing 15 in each groups: Control A, Control B, Control C, and Control D.

On day 14 a booster dose of the protein was injected to Groups A, B, C and D and BSA were injected in control groups. Challenging was done with four different strains of Staphylococci at sub lethal dose to quantify the bacterial vegetation. 10 mice in groups A and Control A were challenged by injecting 3.4×10⁸ cells of ATCC MSSA per mouse via i.v. 10 mice in groups B and Control B were challenged by injecting 3.8×10⁸ cells of ATCC MRSA per mouse via i.v. 10 mice in groups C and Control C were challenged by injecting 3.2×10⁶ cells of clinical MRSA per mouse via i.v. 10 mice in groups D and Control D were challenged by injecting 4×10⁸ cells of ATCC S. epidermidis per mouse i.v. 5 mice in all groups were removed and kept in separate cages at the time of challenging and sera was collected from these mice after 1, 3, 5, 7 and 9 weeks after last immunization to assay specific IgG antibodies against autolysin adhesin. These experiments were also repeated with intraperitoneal inoculation of S. aureus.

### Example 11

### Bacterial Investigation

After 72 hrs of challenging, all mice were sacrificed, dissected and kidneys were removed aseptically for bacterial investigations. Kidneys were washed with ethyl alcohol and sterile PBS to remove surface attached bacteria and homogenized separately in a sterile pestle mortar. To quantify the bacteria 10 fold serial dilution was carried out till a dilution of 10⁻⁵ in sterile PBS. 1 mL of 10⁻³ to 10⁻⁵ dilutions were plated by pour plate method using Vogel Johnson agar (VJ agar) containing 1% potassium tellurite and incubated at 37° C. Colony forming units (cfu) were counted after 36 hr and 48 hrs of incubation. For identification of peritoneal vegetation, 2 mL of sterile PBS was injected into the peritoneal cavity of each mouse, the abdomen of each mouse was massaged for 2 min, and a sample of the lavage fluid was drawn by a syringe and cultured in cooked meat media. Blood culture also was done for identifying any systemic infection. Bacteria were also tested for catalase and coagulase activity.

All the animals used in this study survived S. aureus and S. epidermidis challenge. The number of bacteria in the kidneys from mice vaccinated and non vaccinated controls were as follows: positive controls demonstrated 1.0 up to 8.1×10⁶ cfu per pair of kidney per mouse; mice challenged with 3.4×10⁸ cells of MSSA ATCC 25923 (Group A) demonstrated 0 up to 7×10² cfu per pair of kidney with only 2 out of 10 mice showed mild infection; mice challenged with 3.8×10⁸ cells of MRSA ATCC 33591 (Group B) demonstrated 0 up to 5.1×10⁴ cfu per pair of kidney with only 3 out of 10 mice showing mild infection; mice challenged with 3.2×10⁶ cells of clinical MRSA (A multi drug resistant strain isolated from femur bone of a hospitalized patient) (Group C) demonstrated 0 up to 9×10³ cfu per pair of kidney with only 3 out of 10 mice showing mild infection; and mice challenged with 4×10⁸ cells of S. epidermidis ATCC 12228 (Group D) demonstrated 0 up to 1×10⁴ cfu per pair of kidney with only 2 out of 10 mice showing mild infection. The number of bacteria (cfu)/pair of kidneys in each animal analyzed is shown in Table 1. Sera samples were tested for antibody titer after 1, 3, 5, 7 and 9 weeks after last immunization and demonstrated very high titer even 9 weeks after immunization as shown in FIG. 8.

TABLE 1
Animal infection model
Groups showing cfu from pair of kidneys

| Mice | | Control | | Control | | Control | | Control |
|---|---|---|---|---|---|---|---|---|
| S. No | A | A | B | B | C | C | D | D |
| 1 | 0 | 2 × 10⁶ | 0 | 2 × 10⁶ | 0 | 1.5 × 10⁶ | 0 | 2.5 × 10⁶ |
| 2 | 0 | 1 × 10⁶ | 2 × 10³ | 2.7 × 10⁶ | 0 | 1.6 × 10⁶ | 0 | 2.5 × 10⁶ |
| 3 | 0 | 1 × 10⁶ | 0 | 2.9 × 10⁶ | 0 | 1 × 10⁶ | 0 | 2 × 10⁶ |
| 4 | 0 | 1 × 10⁶ | 0 | 2.8 × 10⁶ | 9 × 10³ | 1.4 × 10⁶ | 0 | 2.4 × 10⁶ |
| 5 | 5 × 10² | 6.1 × 10⁶ | 3.3 × 10⁴ | 2.7 × 10⁶ | 0 | 1 × 10⁶ | 0 | 2.2 × 10⁶ |
| 6 | 7 × 10² | 9 × 10⁶ | 0 | 2.2 × 10⁶ | 8 × 10³ | 1.5 × 10⁶ | 2 × 10³ | 2.4 × 10⁶ |
| 7 | 0 | 1 × 10⁶ | 0 | 2 × 10⁶ | 4 × 10³ | 1.4 × 10⁶ | 0 | 2.5 × 10⁶ |
| 8 | 0 | 1 × 10⁶ | 5.1 × 10⁴ | 2.6 × 10⁶ | 0 | 1.6 × 10⁶ | 0 | 1.9 × 10⁶ |
| 9 | 0 | 1 × 10⁶ | 0 | 1.9 × 10⁶ | 0 | 1.4 × 10⁶ | 0 | 1.8 × 10⁶ |
| 10 | 0 | 8.1 × 10⁶ | 0 | 2 × 10⁶ | 0 | 1.2 × 10⁶ | 1 × 10⁴ | 2 × 10⁶ |

CFU—Colony Forming Unit; 1-10 indicate the Mouse identification.

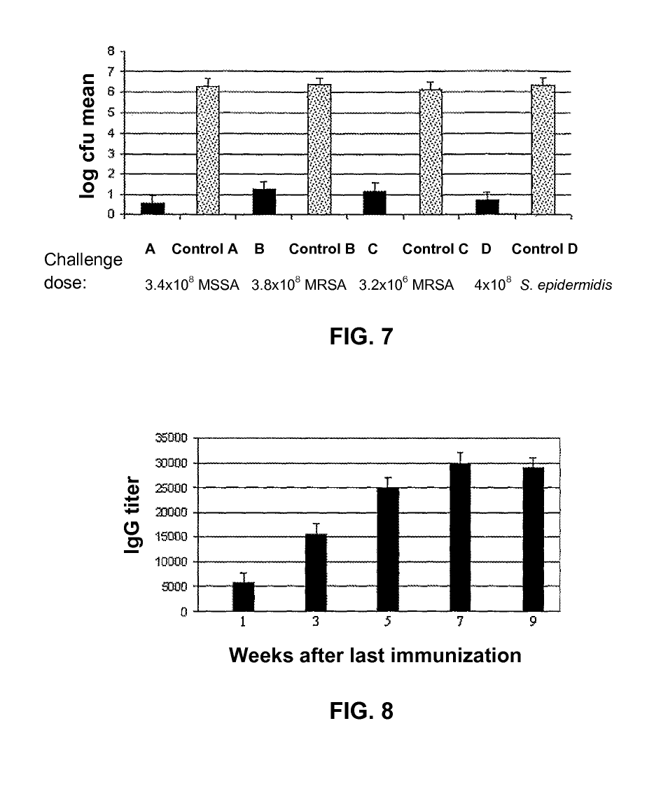

Fisher test was applied to determine the significance of the differences between vaccinated and control groups. The reduction of the bacterial count in kidneys from immunized mice was significant. Kidneys from 80% of the immunized mice in group A, 70% in groups B and C did not show the presence of bacteria after S. aureus challenge and 80% of the immunized mice in group D did not show the presence of bacteria after S. epidermidis challenge as shown in Table 2. Each group log mean CFU is significantly different from the mean CFU of control group as shown in Table 3. From the means, we can see that the infection is much higher among mice in control group than that of the vaccinated groups as shown in FIG. 7. The difference between the controls groups (Control A-D) and the vaccinated groups (Group A-D) was statistically significant as shown in Table 4.

TABLE 2
Frequencies and Percentages of mice based on infection status\

| | |
|---|---|
| | Not |
| Infected | Infected |

| Group | N | % | N | % |
|---|---|---|---|---|
| A | 2 | 20.0 | 8 | 80.0 |
| Control A | 10 | 100.0 | 0 | 0.0 |
| B | 3 | 30.0 | 7 | 70.0 |
| Control B | 10 | 100.0 | 0 | 0.0 |
| C | 3 | 30.0 | 7 | 70.0 |
| Control C | 10 | 100.0 | 0 | 0.0 |
| D | 2 | 20.0 | 8 | 80.0 |
| Control D | 10 | 100.0 | 0 | 0.0 |

TABLE 3
log Mean CFU

| Group | log cfu mean |
|---|---|
| A | 0.55441 |
| Control A | 6.2949 |
| B | 1.25271 |
| Control B | 6.37116 |
| C | 1.14594 |
| Control C | 6.12779 |
| D | 0.7301 |
| Control D | 6.34326 |

TABLE 4
Test of proportions for Vaccinated group and Control group

| | Proportion of mice | Proportion in mice | |
|---|---|---|---|
| Groups | in vaccinated group | in control group | p-value |
| A vs. Control A | 0.20 | 1 | 0.0007 |
| B vs. Control B | 0.30 | 1 | 0.003 |
| C vs. Control C | 0.30 | 1 | 0.003 |
| D vs. Control D | 0.20 | 1 | 0.0007 |

### Example 12

### Immunotherapeutics Development and Assay

Balb/c mice were hyper immunized with the recombinant purified Aaa protein by intraperitoneal route of immunization. Specific hyperimmune sera ware used for purification of IgG fraction by protein G column. The purified IgG was used for passive immunization of Balb/c mice (test group n=8) that were subsequently challenged with 108 cfu of S. aureus ATCC MSSA strain. The control group (control group n=8) was injected with equal volume of the vehicle in lieu of IgG fraction. Both the groups were observed for the mortality for 48 hours. The test group (n=8) survived the S. aureus challenge. However, 100% mortality was observed in control group (n=8) of animals.

This invention is not to be limited to the specific embodiments disclosed herein and modifications for various applications and other embodiments are intended to be included within the scope of the appended claims. While this invention has been described in connection with particular examples thereof, the true scope of the invention should not be so limited since other modifications will become apparent to the skilled practitioner upon a study of the drawings, specification, and following claims.

All publications and patent applications mentioned in this specification are indicative of the level of skill of those skilled in the art to which this invention pertains. All publications and patent applications mentioned in this specification are herein incorporated by reference to the same extent as if each individual publication or patent application mentioned in this specification was specifically and individually indicated to be incorporated by reference.

The invention claimed is:

1. A purified protein selected from the group consisting of:
(a) a protein comprising the amino acid sequence of SEQ ID NO: 3; and
(b) a protein comprising a modified amino acid sequence of SEQ ID NO: 3, wherein amino acids deleterious to protein-protein interactions are replaced through domain mapping,
wherein
said protein, when administered to a patient, prevents or controls Staphylococcal infections.

2. The protein of claim 1, comprising the amino acid sequence of SEQ ID NO: 4.

3. The protein of claim 1, comprising the amino acid sequence of SEQ ID NO: 5.

4. A recombinant DNA construct, comprising: (i) a vector and (ii) at least one nucleic acid fragment encoding amino acid sequence of SEQ ID NO: 3 or a modified amino acid sequence of SEQ ID NO: 3, wherein amino acids deleterious to protein-protein interactions are replaced through domain mapping.

5. The recombinant DNA construct of claim 4, wherein the vector is a prokaryotic plasmid expression vector being cloned in a prokaryotic host.

6. A method for producing the protein of claim 1, comprising the following steps:
(a) culturing a host cell cloned with a recombinant DNA construct comprising (i) a vector and (ii) at least one nucleic acid encoding amino acid sequence of SEQ ID NO: 3 or a modified amino acid sequence of SEQ ID NO: 3, wherein amino acids deleterious to protein-protein interactions are replaced through domain mapping, wherein the vector is a prokaryotic plasmid expression vector being cloned in a prokaryotic host;
(b) harvesting cells cultured in step (a) and isolating a recombinant protein therefrom; and
(c) purifying the recombinant protein by solubilizing the recombinant protein under denaturing conditions using at least one of the following denaturing agents: urea, or guanidine hydrochloride, in the range from 0.1 M to 12 M, and further capturing the recombinant protein on an adjuvant.

7. The method of claim 6, wherein the adjuvant is selected from the group consisting of: aluminum hydroxide, aluminum phosphate, calcium phosphate, or mineral oil.

8. A pharmaceutical composition, comprising: a pharmaceutically and physiologically acceptable carrier and the purified protein of claim 1, wherein the purified protein is provided in an effective amount in the range of from 1 to 1000 mg.

9. The pharmaceutical composition of claim 8, wherein the purified protein is provided in an effective amount in the range of from 5 to 500 mg.

10. The pharmaceutical composition of claim 8, wherein the purified protein is provided in an effective amount in the range of from 10 to 100 mg.

11. The pharmaceutical composition of claim 8, wherein said purified protein of claim 1 is conjugated either through a linker or without a linker to said pharmaceutically and physiologically acceptable carrier; and said pharmaceutically and physiologically acceptable carrier is a peptide or a polysaccharide.

12. The pharmaceutical composition of claim 8, further comprising at least one carrier buffer selected from the group consisting of: a phosphate buffer, a phosphate-citrate buffer, and further comprising an added adjuvant selected from the group consisting of: aluminum hydroxide, aluminum phosphate, calcium phosphate, and mineral oil.

13. The pharmaceutical composition of claim 8, further comprising at least one pharmaceutically and physiologically accepted stabilizing agent selected from the group consisting of: a polyol, glycerol, human serum albumin, a sugar, and an amino acid, wherein said stabilizing agent is provided in the range of from 0.05% to 5% by weight with respect to the protein.

14. A formulation comprising a recombinant plasmid and a pharmaceutically acceptable carrier, said plasmid consisting of at least one nucleotide coding sequence of a Staphylococcus aureus protein antigen as claimed in claim 1 including transcriptional and translational regulatory sequences operably linked to said nucleotide sequence for expressing said polypeptide in a mammal.

15. A method of immunodiagnosing a Staphylococcal infection, comprising: (a) combining a sample suspected of containing antibodies to Staphylococcus with the pharmaceutical composition of claim 8 to form a reaction mixture, (b) allowing said mixture to incubate for a time sufficient for binding between said antibodies and said protein to occur and form bound immunocomplexes, (c) separating the bound immunocomplexes from unbound reagents, and (d) detecting the presence of the bound complexes by means of an effective label.

16. A method for treating or preventing a Staphylococcal infection, comprising (a) administering to a patient the pharmaceutical composition of claim 8.

17. The method of claim 16, wherein the pharmaceutical composition of claim 8 is administered by at least one route of administration selected from intramuscular, intradermal, subcutaneous, intravenous, oral, or intranasal.

18. The method of claim 16, wherein the patient is a human, a bovine mammal, a renal dialysis patient, a patient undergoing surgery, a patient with indwelling medical device, a subject with traumatic wounds, a symptomatic carrier, or an asymptomatic carrier.


SEQUENCE LISTING (8 sequences)

SEQ ID NO: 1 - DNA, 1060 bp, Staphylococcus aureus
ggaaagttaagcaagaggaggattttaaagtgcaaaaaaaagtaattgcagctattattgggacaagcgcgattagcgctgttgcggcaactcaagcaaatgcggctacaactcacacagtaaaaccgggtgaatcagtgtgggcaatttcaaataagtatgggatttcgattgctaaattaaagtcattaaacaatttaacatctaatctaattttcccaaaccaagtactaaaagtatctggctcaagtaattctacgagtaatagtagccgtccatcaacgaactcaggtggcggatcatactacacagtacaagcaggcgactcattatcattaatcgcatcaaaatatggtacaacttaccaaaacattatgcgacttaatggtttaaataatttctttatttatccaggtcaaaaattaaaagtatcaggtactgctagctcaagtaacgctgcgagcaatagtagccgtccatcaacgaactcaggtggcggatcatactatacagtacaagcaggtgactcattgtcattaatcgcatcaaaatatggtacaacttatcaaaaaattatgagcttaaatggcttaaataatttctttatatatccgggtcaaaaattgaaagtaactggtaatgcatctacgaactcaggatctgcaacaacgacaaatagaggttacaatacaccagtattcagtcaccaaaacttatatacatggggtcaatgtacatatcatgtatttaatcgtcgtgctgaaattggtaaaggtattagtacttattggtggaatgctaataactgggataacgcagcggcagcagatggttacactatcgacaatagacctactgtaggttctatcgctcaaacagatgtaggttattatggtcatgttatgtttgtagaacgtgtaaataacgatggtagtattttagtttcagaaatgaactattcagctgcaccaggtattttaacttacagaacggtaccagcttaccaagtaaataattatagatatattcactaaagtcttacgtatataaatatataatg

SEQ ID NO: 2 - protein, 334 aa, Staphylococcus aureus
MQKKVIAAIIGTSAISAVAATQANAATTHTVKPGESVWAISNKYGISIAKLKSLNNLTSNLIFPNQVLKVSGSSNSTSNSSRPSTNSGGGSYYTVQAGDSLSLIASKYGTTYQNIMRLNGLNNFFIYPGQKLKVSGTASSSNAASNSSRPSTNSGGGSYYTVQAGDSLSLIASKYGTTYQKIMSLNGLNNFFIYPGQKLKVTGNASTNSGSATTTNRGYNTPVFSHQNLYTWGQCTYHVFNRRAEIGKGISTYWWNANNWDNAAAADGYTIDNRPTVGSIAQTDVGYYGHVMFVERVNNDGSILVSEMNYSAAPGILTYRTVPAYQVNNYRYIH

SEQ ID NO: 3 - protein, 316 aa, Staphylococcus aureus
MATTHTVKPGESVWAISNKYGISIAKLKSLNNLTSNLIFPNQVLKVSGSSNSTSNSSRPSTNSGGGSYYTVQAGDSLSLIASKYGTTYQNIMRLNGLNNFFIYPGQKLKVSGTASSSNAASNSSRPSTNSGGGSYYTVQAGDSLSLIASKYGTTYQKIMSLNGLNNFFIYPGQKLKVTGNASTNSGSATTTNRGYNTPVFSHQNLYTWGQCTYHVFNRRAEIGKGISTYWWNANNWDNAAAADGYTIDNRPTVGSIQTDVGYYGHVMFVERVANNDGSILVSEMNYSAAPGILTYRTVPAYQVNNYRYIHHHHHHH

SEQ ID NO: 4 - protein, 150 aa, Staphylococcus aureus
MATTHTVKPGESVWAISNKYGISIAKLKSLNNLTSNLIFPNQVLKVSSGGGSHTVKPGESVWAISNKYGISIAKLKSLNNLTSNLIFPNQVLKVSSGGGSVGYYGHVMFVERVNNDGSILVSEMNYSAAPGILTYRTVPAYQVNNYRYIH

SEQ ID NO: 5 - protein, 127 aa, Staphylococcus aureus
MQKKVIAAIIGTSAISAVAATQANAYYTVHAGDSLSLIASKYGTTYQNIMRLNGGTASSSNAASNSSRPSTNSGGGSVGYYGHVMFVERVNNDGSILVSEMNYSAAPGILTYRTVPAYQVNNYRYIH

SEQ ID NO: 6 - protein, 324 aa, Staphylococcus epidermidis
MQKKYITAIIGTTALSALASTHAQAATTHTVKSGESVWSISHKYGISIAKLKSLNGLTSNLIFPNQVLKVSGSSSRATSTNSGTVYTVKAGDSLSSIAAKYGTTYQKIMQLNGLNNYLIFPGQKLKVSGKATSSSRAKASGSSGRTATYTVKYGDSLSAIASKYGTTYQKIMQLNGLTNFFIYPGQKLKVPGGSSSSSSSNNTRSNGGYYSPTFNHQNLYTWGQCTWHVFNRRAEIGKGISTYWWNANNWDNASAADGYTIDYRPTVGSIAQTDAGYYGHVAFVERVNSDGSILVSEMNWSAAPGNMTYRTIPAYQVRNYKFIH

SEQ ID NO: 7 - DNA, 35 bp, Artificial Sequence
  Synthetic forward primer for amplification of gene fragment corresponding to mature protein of S. aureus autolysin adhesin (Aaa) gene by PCR
cgagctccatatggctacaactcacacagtaaaac

SEQ ID NO: 8 - DNA, 62 bp, Artificial Sequence
  Synthetic reverse primer for amplification of gene fragment corresponding to mature protein of S. aureus autolysin adhesin (Aaa) gene by PCR
cgctcgagggatccttattagtgatggtgatggtgatggtgaatatatctataattatttac